(19) United States
(12) Patent Application Publication
(10) Pub. No.: US 2011/0284412 A1
(43) Pub. Date: Nov. 24, 2011

(54) PHARMACEUTICAL POLYPEPTIDE DRY POWDER AEROSOL FORMULATION AND METHOD OF PREPARATION

(76) Inventors: Cecily B. Lalor, San Carlos, CA (US); Jeffrey S. Tepper, San Carlos, CA (US); Wayne A. Froland, Alameda, CA (US)

(21) Appl. No.: 12/912,398

(22) Filed: Oct. 26, 2010

Related U.S. Application Data
(63) Continuation of application No. 12/571,347, filed on Sep. 30, 2009.

(51) Int. Cl. A61B 19/02

(52) U.S. Cl. 206/438

(57) ABSTRACT

Dispersible powder compositions suitable for inhalation are disclosed, the compositions including a human interleukin mutein (mhIL-4).

### CROSS REFERENCE TO RELATED APPLICATIONS

This application is a continuation application of U.S. patent application Ser. No. 12/571,347 filed Sep. 30, 2009, now pending, which claims the benefit of International Application No. PCT/US2008/069889 filed Jul. 11, 2008, now pending; which claims the benefit under 35 USC §119(e) to U.S. Application Ser. No. 60/959,267 filed Jul. 11, 2007, now abandoned. The disclosure of each of the prior applications is considered part of and is incorporated by reference in the disclosure of this application.

### BACKGROUND OF THE INVENTION

1. Field of the Invention

The present invention relates generally to the methods and compositions useful for treating pulmonary (respiratory) disorders, including allergic diseases such as asthma, and more specifically, dry powder aerosol compositions comprising mutiens of human IL-4.

2. Background Information

Interleukin-4 (IL-4) and Interleukin-13 (IL-13) are pleiotropic cytokines with a broad spectrum of similar biological effects on several target cells important in the pathogenesis of several lung diseases. The redundancy in effects associated with the binding and signaling of these two cytokines can be explained by their sharing of common receptor components. Recently, certain antagonistic and partially antagonistic properties have been observed in human mutant IL-4 (mIL-4) proteins in which the amino acid(s) occurring naturally in the wild type at one or more of positions 120, 121, 122, 123, 124, 125, 126, 127 or 128 have been replaced with one or more natural amino acids. Thus, these mIL-4 proteins have been described as valuable therapeutic agents for use as medicaments in treating overshooting or falsely regulated immune reactions and autoimmune diseases.

To adequately achieve the desirable physiological effects of such mutant proteins, a formulation and method of administering the protein in its active form is required. Although systemic, but not oral, delivery is feasible, mIL-4 proteins have a short half-life necessitating frequent injection. Because systemic delivery is not ideal using these mutant proteins, lung drug delivery systems should be tried. Furthermore, there may be a selective advantage to delivering the drug to site of disease.

### SUMMARY OF THE INVENTION

According to embodiments of the present invention, pharmaceutical compositions comprising a mIL-4 mutant protein suitable for long-term inhalation administration to a patient in need thereof are provided. In some embodiments, there are provided dispersible powder compositions suitable for inhalation by a patient in need thereof, the composition comprising a human interleukin mutein (mIL-4), wherein a glass transition temperature of the composition is at least 50° C. higher than a storage temperature at which the composition is stored. A total storage period of the composition is at least two years, and the composition retains at least 80% of the original specific activity after the composition is stored at the storage temperature over a period of three months.

According to other embodiments of the present invention, in addition to mIL-4, the composition also includes a buffer, such as a citrate, an acetate, a lactate, or a succinate, a maleate, or a tartarate, and a stabilizing agent, such as a carbohydrate, e.g., sucrose, mannitol, or trehalose. The composition may further optionally comprise an excipient, such as an amino acid (e.g., leucine) or a poly(amino acid); the composition may also optionally comprise salts of magnesium, e.g., magnesium sulfate.

According to other embodiments of the present invention, manufacturing methods are provided permitting the production of pharmaceutical compositions of sufficient purity that are easily dispersable and of respirable size, such that the pharmaceutical has a high deposited fraction in the lung, the method allowing to maintain high percentage of pharmaceutical activity.

According to other embodiments of the present invention, the composition may be incorporated into a kit for the use by a patient in need of the composition, the kit comprising the composition, an inhaling means for inhalation by the patient, and a storage means for storing the composition. In some embodiments, the storage means comprises a primary capsule adapted to fit the inhaling means, and a secondary storage container. The kit further optionally includes a label affixed to the storage means and providing the patient with instructions for use.

### BRIEF DESCRIPTION OF THE DRAWINGS

FIG. 1A shows the nucleic acid and amino acid sequences for wt IL-4.

FIG. 1B shows the nucleic acid and amino acid sequences for a mutant mIL-4.

FIG. 2 shows schematically a delivery kit according to some embodiments of the present invention.

FIG. 3 shows: high pressure liquid chromatography chromatograms for one formulation according to some embodiments of the present invention.

FIG. 4 shows SDS PAGE gels for feedstock solution and spray dry powders for formulations according to some embodiments of the present invention.

FIG. 5 shows Differential Scanning calorimetry (DSC) thermograms of spray dried mIL-4 formulations according to some embodiments of the present invention.

FIG. 6 shows DSC thermograms of spray dried mIL-4 formulations according to some embodiments of the present invention.

FIG. 7 shows particle size distribution of a formulation according to some embodiments of the present invention.

FIG. 8 is an exploded perspective view of the inhaler device that may be used for administering powder formulations according to some embodiments of the present invention.

FIG. 9 is a further perspective view of the inhaler device that may be used for administering powder formulations according to some embodiments of the present invention, wherein the inhaler device is shown in an open condition thereof, i.e. in the capsule loading position thereof.

FIG. 10 is a view similar to that of FIG. 9, but illustrating the inhaler device according to the present invention during the use thereof.

FIG. 11 is an elevation cross-sectional view of the inhaler device that may be used for administering powder formulations according to some embodiments of the present invention, wherein the inhaler device is shown with a capsule arranged therein, but in a non perforated condition.

FIG. 12 is a view similar to that of FIG. 11, but illustrating the inhaler device according to the, present invention during the capsule perforating operation.

FIG. 13 is a top plan view, as partially cross-sectioned, of the inhaler device that may be used for administering powder formulations according to some embodiments of the present invention.

FIG. 14 is a plasma concentration versus time profile of a IL-4 mutein dry powder composition.

### DETAILED DESCRIPTION OF THE INVENTION

The term pulmonary and respiratory are defined as having to do with the lungs. The term “mutein” is defined as referring to any protein arising as a result a site-directed amino acid substitution to any protein created by a person skilled in the art. “Glycosylation” refers to the addition of glycosyl groups to a protein to form a glycoprotein. As such, the term includes both naturally occurring glycosylation and synthetic glycosylation, such as the linking of a carbohydrate skeleton to the side chain of an asparagine residue (“N-glycosylation”) or the coupling of a sugar, preferably N-acetylgalactosamine, galactose or xylose to serine, threonine, 4-hydroxyproline or 5-hydroxylysine (O-glycosylation).

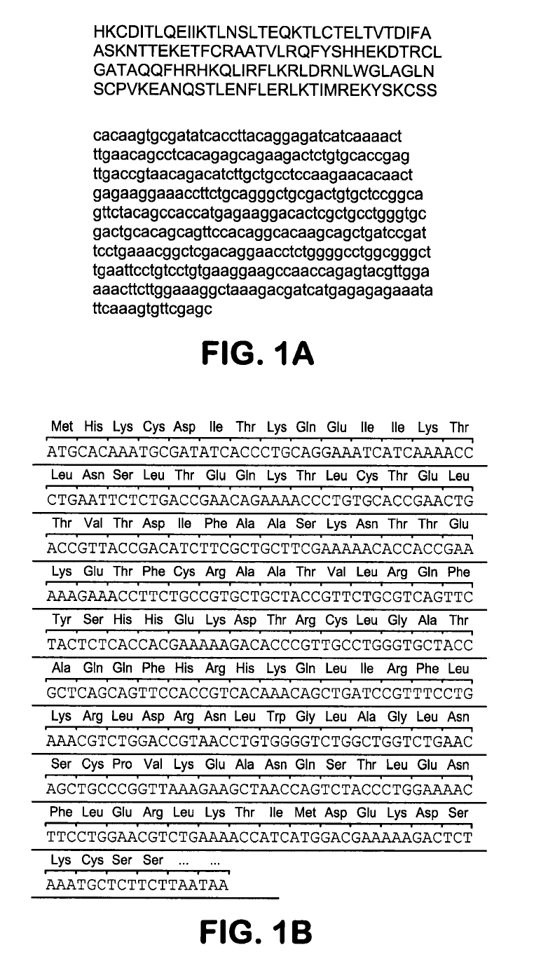

Mutants of human IL-4 that function as agonists are known in the art. The terms “IL-4 mutein,” “IL-4 mutant,” “mIL-4,” “human mutant IL-4 protein,” “mhIL-4,” “modified human IL-4 receptor antagonist,” “IL-4RA,” “IL-4 antagonist,” and equivalents thereof are used interchangeably and are within the scope of the invention. These polypeptides may optionally include additional residues beyond the “N” and “C” termini of the wild type protein. These polypeptides and functional fragments thereof refer to polypeptides wherein specific amino acid substitutions to the wildtype human IL-4 protein (“wt IL-4”; FIG. 1A) have been made. These polypeptides include the mhIL-4 compositions of the present invention, which are administered to a subject in need of treatment for asthma, for example. In particular, the exemplary mhIL-4 of the present invention, include at least the R121D/Y124D pair of substitutions with an N-terminal methionine (“IL-4RA” or “met-R121D/Y124D”), as shown in FIG. 1B.

The term a “functional fragment” is defined for the purposes of the present application as a polypeptide which has IL-4 antagonistic activity, including smaller peptides. These and other aspects of mIL-4 of modification of mIL-4 are described in U.S. Pat. Nos. 6,313,272; and 6,028,176, the entire contents of which are incorporated herein by reference.

The terms “wild type IL-4” or “wtIL-4” and equivalents thereof are used for the purposes of the present application interchangeably and mean human Interleukin-4, native or recombinant, having the 129 normally occurring amino acid sequence of native human IL-4, as disclosed in U.S. Pat. No. 5,017,691, the entire contents of which is incorporated herein by reference. Further, the modified human mIL-4 receptor antagonists, which do not cause signaling at the cognate receptor to which it binds as described herein, may have various insertions and/or deletions and/or couplings to a non-protein polymer, and are numbered in accordance with the wtIL-4, which means that the particular amino acid chosen is that same amino acid that normally occurs in the wtIL-4. Accordingly, one skilled in the art will appreciate that the normally occurring amino acids at positions, for example, 121 (arginine), 124 (tyrosine), and/or 125 (serine), may be shifted in the mutein. Thus, an insertion of a cysteine residue at amino acid positions, for example, 38, 102 and/or 104 may be shifted on the mutein. However, the location of the shifted Ser (S), Arg (R), Tyr (Y) or inserted Cys (C) can be determined by inspection and correlation of the flanking amino acids with those flanking Ser, Arg, Tyr or Cys in wilL-4.

The term a “primary particle size” is defined for the purposes of the present application as the size of the particle as measured by various techniques such as laser diffraction, scanning electron microscopy and sedimentation.

The term “aerodynamic” is defined for the purposes of the present application as the diameter of a sphere of unit density which has the same settling velocity in air as the aerosol particle being measured. Aerodynamic diameter is measured by a cascade impactor. The term “mass median aerodynamic diameter” or “MMAD” is defined as the median of the distribution of mass with respect to aerodynamic diameter. The median aerodynamic diameter and the geometric standard deviation are used to describe the particle size distribution of an aerosol, based on the mass and size of the particles. According to such a description, fifty percent of the particles by mass will be smaller than the median aerodynamic diameter, and fifty percent of the particles will be larger than the median aerodynamic diameter.

Further, the DNA sequence encoding human mIL-4 may or may not include DNA sequences that encode a signal sequence. Such signal sequence, if present, should be one recognized by the cell chosen for expression of the mIL-4 mutein. It may be prokaryotic, eukaryotic or a combination of the two. It may also be the signal sequence of native IL-4. The inclusion of a signal sequence depends on whether it is desired to secrete the mIL-4 mutein from the recombinant cells in which it is made. If the chosen cells are prokaryotic, it generally is preferred that the DNA sequence not encode a signal sequence but include an N-terminal methionine to direct expression. If the chosen cells are eukaryotic, it generally is preferred that a signal sequence be encoded and most preferably that the wild-type IL-4 signal sequence be used, as disclosed in U.S. Pat. No. 6,028,176, incorporated herein by reference.

In one illustrative example, a mutant human mIL-4 protein of the invention includes the amino acid sequence of wild-type hIL-4 with modifications, wherein a first modification is replacement of one or more of the amino acids occurring in the wild-type hIL-4 protein at positions 121, 124 or 125 with another natural amino acid, and further optionally comprising an N-terminal methionine. In another example, the mutant protein further includes a second modification selected from the group consisting of

i) the modification of the C-terminus therein;

ii) the deletion of potential glycosylation sites therein;

iii) the coupling of the protein to a non-protein polymer,

iv) at least one amino acid substitution selected from the group consisting of substitutions at positions 13, 16, 81 and 89,

and any combination thereof with mIL-4 protein being an antagonist of wild-type hIL-4 and further optionally comprising an N-terminal methionine.

In yet another example, the mutant protein includes a first modification of the protein that includes substitutions R121D and Y124D, numbered in accordance with the wild-type hIL-4.

The terms “subject” or “patient” as used herein refer to any individual or patient to which the subject methods are performed. Typically, the subject is human, although as will be appreciated by those in the art, the subject may be an animal. Thus, non human primates (including monkeys, chimpanzees, orangutans and gorillas) are included within the definition of subject.

The terms “administration” or “administering” are defined to include an act of providing a compound or pharmaceutical composition of the invention to a subject in need of treatment. The term “therapeutically effective amount” or “effective amount” means the amount of a compound or pharmaceutical composition that will elicit the biological or medical response of a tissue, system, animal or human that is being sought by the researcher, veterinarian, medical doctor or other clinician.

The term a “powder” is defined for the purposes of the present application as a solid substance formulated as finely divided solid particles that are smaller than about 10 micrometers in dimension, such as a solid substance formulated as finely divided dry solid particles that are smaller than about 6 micrometers in dimension.

The term “glass transition temperature” is defined for the purposes of the present application as an approximate midpoint in the temperature range at which a reversible change occurs in a substance when it is heated to a certain temperature and undergoes a transition from glassy condition to elastomeric condition. Glass transition (Tg) is determined using differential scanning calorimetry (DSC). The definition of Tg is always arbitrary and there is no present international convention.

Asthma is a chronic inflammatory or an allergic disorder of the airways in which many cells and cellular elements play a role, in particular, mast cells, eosinophils, T lymphocytes, airway macrophages, neutrophils, and epithelial cells. In susceptible individuals, this inflammation causes recurrent episodes of wheezing, breathlessness, chest tightness, and coughing, particularly at night or in the early morning. These episodes are usually associated with widespread but variable airflow obstruction that is often reversible either spontaneously or with treatment. The inflammation also causes excessive mucus secretion and an associated increase in the existing bronchial hyperresponsiveness to a variety of stimuli.

According to embodiments of the invention, there are provided pharmaceutical dispersible dry powder compositions exhibiting good temperature and structural stability, including resistance to moisture and aggregation. The compositions include a therapeutic agent comprising a human interleukin-4 mutein (mIL-4). The powder is formed by particles typically having the mean value of a primary particle size and/or aerodynamic diameter size less than about 10 μm, such as between about 2 μm and about 6 μm, for example, between about 2 μm and about 4 μm. The geometric standard deviation in the particle size distribution is between about 1 and 3, such as between about 1.5 and 2.5.

A variety of embodiments can generally characterize and illustrate the features of the instant invention. In one embodiment, there is provided a dispersible powder composition suitable for inhalation by a patient in need thereof, the composition including a therapeutic agent comprising a human interleukin mutein (mhIL-4), wherein a glass transition temperature of the composition is at least 50° C. higher than a storage temperature at which the composition is stored, and wherein the composition retains at least 80% of the original specific activity after the composition is stored at the storage temperature over a period of three months.

According to another embodiment, there is provided a dispersible powder composition suitable for inhalation by a patient in need thereof, the composition including a therapeutic agent comprising a mutant human interleukin-4 (mIL-4) protein consisting of the amino acid sequence of wild-type hIL-4 with two modifications, wherein the first modification is selected from the group consisting of the replacement of one or more of the amino acids occurring in the wild-type hIL-4 protein at positions 121, 124, or 125 with another natural amino acid, and the second modification is at least one modification selected from the group consisting of the modification of the C-terminus therein; the deletion of potential glycosylation sites therein; and/or the coupling of the proteins to a non-protein polymer, and any combination thereof; at least one amino acid substitution selected from the group consisting of substitutions at positions 13, 16, 81 and 89, and any combination thereof, with mIL-4 protein being an antagonist of wild-type hIL-4 and further optionally an N-terminal methionine. Such a composition may further have a glass transition temperature of at least 50° C. higher than a storage temperature at which the composition is stored, wherein the composition retains at least 80% of the original specific activity after the composition is stored at the storage temperature over a period of three months.

In some embodiments, any compositions described above, in addition to mhIL-4 or mIL-4 described above may further include a buffer, such as a citrate, an acetate, a lactate, a tartarate, a succinate, or a maleate, and a stabilizing agent, such as a carbohydrate, e.g., sucrose, mannitol, or trehalose, or a magnesium salt, e.g., magnesium sulfate. In some embodiments, any composition, whether it does or does not include a buffer and/or a stabilizing agent, may further comprise, in addition to mhIL-4 described above, an excipient selected from a group consisting of an amino acid, e.g., leucine, or a poly(amino acid).

Compositions of any embodiment discussed above has the glass transition temperature of the composition is at least 75° C. higher than the storage temperature (i.e., the temperature at which the composition is stored, which may be room temperature or below, e.g., between about 2° C. and 8° C., alternatively, the storage temperature between about 2° C. and 8° C. during a first portion of the storage period, and room temperature during a second portion of the storage period), such as at least 100° C. higher than the storage temperature, and after the total storage period of at least two years, compositions of any embodiment discussed above retain at least 95% of the original specific activity after the expiration of a total storage period, for example retaining at least 98% of the original specific activity.

Compositions of any embodiment discussed above have the mass concentration of the therapeutically active material in the composition is between about 10% and about 98%, such as between about 10% and about 75%, for example, between about 10% and about 60%.

Compositions of any embodiment discussed above have the moisture content between about 1% and about 10%, such as between about 1% and about 5, for example, between about 1% and about 3%.

Compositions of any embodiment discussed above have the degree of aggregation of about 3% or less after the expiration of a total storage period of at least two years, such as about 1% or less, or about 0%.

Compositions of any embodiment discussed above have the degree of oxidation, relative to the drug substance, of the therapeutically active material after the expiration of a total storage period of the composition of about 5% or less, wherein the total storage period is at least two years. For example, such degree of oxidation may be about 3% or less, or about 2%.

Compositions of any embodiment discussed above is a powder formed by particles having the mean value of diameter less than about 10 μm, for example between about 2 μm and about 6 μm, such as between about 2 μm and about 4 μm.

Compositions of any embodiment discussed above include particles having the geometric standard deviation in the particle size between about 1 and 3, for example, between about 1.5 and 2.5.

Compositions of any embodiment discussed above provide the emitted dose of the composition, when inhaled by the patient, that is about 70 mass % or higher, such as about 80 mass % or higher, for example, about 90 mass % or higher.

Compositions of any embodiment discussed above provide, when inhaled by a patient, the deposited fraction of the particles having the value of diameter not exceeding about 5 μm that is between about 25 and about 60 mass %, such as between about 40 and about 60 mass %, for example, between about 50 and about 60 mass %.

Compositions of any embodiment discussed above have the pH value that is between about 3 and 6, such as between about 4 and 5.

Compositions of any embodiment discussed above have a nominal dose of the active substance between about 0.3 and 30 mg, for example, between about 0.3 and 5 mg, such as between about 0.5 and 3 mg.

In compositions of any embodiment discussed above, the IL-4 mutein comprises the amino acid sequence of wild-type hIL-4 with modifications, wherein a first modification is replacement of one or more of the amino acids occurring in the wild-type hIL-4 protein at positions 121, 124 or 125 with another natural amino acid, and further optionally comprising an N-terminal methionine. Furthermore, the IL-4 mutein may comprise a second modification selected from the group consisting of the modification of the C-terminus therein; the deletion of potential glycosylation sites therein; the coupling of the protein to a non-protein polymer, at least one amino acid substitution selected from the group consisting of substitutions at positions 13, 16, 81 and 89, and any combination thereof. The first modification may include substitutions R121D and Y124D numbered in accordance with the wild-type hIL-4, or substitutions R121D and Y124D numbered in accordance with the wild-type hIL-4 and an N-terminal methionine.

Compositions of any embodiment discussed above may be prepared by freeze drying, spray drying, and freeze spray drying, and may further optionally include milling or lyophilization with milling.

There are further provided methods of treatment of a disease, comprising administering to a patient in need of such treatment a therapeutically effective amount of a composition of any embodiment discussed above.

In some embodiments, there are provided inhaler devices, comprising an inhaler body defining a recess for holding therein a capsule containing the dispersible powder composition of any embodiment discussed above, and a nosepiece communicating with said capsule, wherein the inhaler device further comprises perforating means associated with the inhaler body and adapted to perforate said capsule to allow an outside air flow to be mixed with the dispersible powder composition for inhalation through said nosepiece. Such devices are designed to ensure that when the properly formulated dry powder composition is inhaled by the patient, the emitted dose of the composition is about 70 mass % or higher. In some embodiments, the perforating means in the inhaler devices comprise perforating needles for transversely sliding against the biasing of resilient elements and operating between an abutment element, rigid with said inhaler body and a corresponding operating push-button element, each perforating needle having a contour including a beveled tip, for facilitating a perforation of a coating of the capsule.

In further embodiments, the nosepiece in the inhaler devices is movable with respect to the inhaler body to provide at least two operating condition, the two operating conditions comprising an open condition in which the recess for the capsule is accessible to engage therein a new capsule or to withdraw therefrom a used capsule, and a closed use condition in which said inhaler nosepiece is snap locked. The nosepiece may be further locked in its closure position by a snap locking means including a hook portion of a flange of the nosepiece, having a corresponding ridge formed inside a latching seat formed in the inhaler body. In further embodiments, the flange of the inhaler nosepiece may comprise a peg which is engageable in a hole formed in the inhaler body. In further embodiments, the hole may define a longitudinal slot adapted to allow a transversal tooth of said peg to pass through the slot, and the hole comprises a bottom annular recess adapted to allow the tooth to slide in, thereby allowing said peg to be engaged in said hole. In further embodiments, the pin may be rotatable in the hole and the nosepiece is rotatable with respect to the inhaler body. In further embodiments, the recess for the capsule of the inhaler body of the device may communicate with the outside through a perforated plate or grid provided in the inhaler nosepiece at the flange and is adapted to separate the capsule recess from a duct of the nosepiece, the capsule recess having a bottom communicating with the outside through one or more air inlet holes.

In some embodiments, there are provided kits comprising the dispersible powder composition of any embodiment discussed above, an inhaling means for inhalation by the patient, and a storage means for storing the composition, the storage means comprising a primary container and a secondary storage container, and optionally further including a label contained in affixed to the storage means and providing the patient with instructions for use, with the further proviso that the primary capsule is adapted to fit the inhaling means. In further embodiments, the composition used with the kit is in form of capsules, each capsule containing between about 5 and 25 mg of the composition, such as between about 5 and about 20 mg of the composition, for example, between about 10 and about 20 mg of the composition.

The quantity of the active substance in the dry powder compositions of the present invention is nominally between about 0.3 and 30 mg, such as between about 0.3 and 5 mg, for example, about 0.5 and 3 mg. In one embodiment, the minimum quantity of the active substance in the dry powder compositions of the present invention can be about 0.3 mg; in other embodiments such minimum quantity can be about 0.5 mg, about 0.7 mg, about 0.75 mg, about 1 mg, about 1.5 mg, or about 2 mg. In one embodiment, the maximum quantity of the active substance in the dry powder compositions of the present invention can be about 3 mg; in other embodiments such maximum quantity can be about 5 mg, about 7.5 mg, about 10 mg, about 12 mg, about 15 mg, or about 30 mg. Overall, the mass concentration of mIL-4 in the composition can be between about 10% and about 98%, such as between about 10% and about 75%, for example, between about 10% and about 60%. In another embodiment, the mass concentration of mIL-4 in the composition can be between about 5% and about 98%, such as between about 5% and about 75%, for example, between about 5% and about 60%, or between about 5% and about 50%, or about 5%, or about 10%, or about 15%, or about 30%. In this context, the percentage (by weight) of the m-IL4 compound refers to the amount of the free compound, excluding the weight of counterion(s) that may be present.

Dry powder compositions of the invention typically, but not necessarily, include at least one physiologically acceptable carrier. For example, the dry powder composition can include one or more excipients, and/or any other component that improves the effectiveness of the mIL-4 compound. Such excipients may serve simply as bulking agents when it is desired to reduce the active agent concentration in the powder which is being delivered to a patient. Such excipients may also serve to improve the dispersability of the powder within a powder dispersion device in order to provide more efficient and reproducible delivery of the active agent and to improve the handling characteristics of the active agent (e.g., flowability and consistency) to facilitate manufacturing and powder filling. In particular, the excipient materials can often function to improve the physical and chemical stability of the mIL-4, to minimize the residual moisture content and hinder moisture uptake, and to enhance particle size, degree of aggregation, surface properties (e.g., rugosity), ease of inhalation, and targeting of the resultant particles to the deep lung.

Pharmaceutical excipients and additives useful in the practice of the present invention include, but are not limited to, proteins, peptides, amino acids, lipids, polymers, and carbohydrates (e.g., sugars, including monosaccharides, di-, tri-, tetra-, and oligosaccharides; derivatized sugars such as alditols, aldonic acids, esterified sugars; and polysaccharides or sugar polymers), which may be present singly or in combination. Exemplary protein excipients include serum albumin such as human serum albumin (HSA), recombinant human albumin (rHA), gelatin, and casein. Representative amino acid/polypeptide components include alanine, glycine, arginine, betaine, histidine, glutamic acid, aspartic acid, cysteine, lysine, leucine, proline, isoleucine, valine, methionine, phenylalanine, aspartame. Polyamino acids of the representative amino acids such as di-leucine and tri-leucine are also suitable for use with the present invention.

Carbohydrate excipients suitable for use in the invention include, for example, monosaccharides such as fructose, maltose, galactose, glucose, D-mannose, and sorbose; disaccharides, such as lactose, sucrose, trehalose, cellobiose; polysaccharides, such as raffinose, melezitose, maltodextrins, dextrans, and starches; and alditols, such as mannitol, xylitol, maltitol, lactitol, xylitol sorbitol (glucitol), and myoinositol.

The mIL-4 dry powder compositions may also include a buffer or a pH adjusting agent; typically, the buffer is a salt prepared from an organic acid or base. Representative buffers include organic acid salts such as salts of citric acid, ascorbic acid, gluconic acid, carbonic acid, tartaric acid, succinic acid, acetic acid, or phthalic acid; Tris, tromethamine hydrochloride, or phosphate buffers.

Additionally, the mIL-4 dry powder compositions useful in the practice of the invention may include polymeric excipients/additives such as polyvinylpyrrolidones, hydroxypropyl methylcellulose, methylcellulose, ethylcellulose, Ficolls (a polymeric sugar), dextran, dextrates (e.g., cyclodextrins, such as 2-hydroxypropyl-beta-cyclodextrin, hydroxyethyl starch), polyethylene glycols, pectin, salts (e.g., sodium chloride), antioxidants, antistatic agents, surfactants (e.g., polysorbates such as “TWEEN 20” and “TWEEN 80”, lecithin, oleic acid, benzalkonium chloride, and sorbitan esters), lipids (e.g., phospholipids, fatty acids), steroids (e.g., cholesterol), and chelating agents (e.g., EDTA). Other examples of pharmaceutical excipients and/or additives suitable for use in the mIL-4 dry powder compositions are listed in “Remington: The Science & Practice of Pharmacy”, 19th ed., Williams & Williams, (1995), and in the “Physician's Desk Reference”, 52nd ed., Medical Economics, Montvale, N.J. (1998), the disclosures of which are herein incorporated by reference.

The dry powder compositions of the present invention further include additional components, such as a buffer, a stabilizing agent, and/or a bulking excipient. The examples provided some preferred amounts and/or combinations of these agents.

The buffer is typically selected from at least one of a citrate, an acetate, a lactate, and/or a tartarate. According to embodiments of the invention, the stabilizing agent typically comprises at least one carbohydrate, such as sucrose, mannitol, and/or trehalose, and the excipient is an amino acid, such as leucine, or a poly(amino acid), or the stabilizing agent may be a salt of magnesium, such as magnesium sulfate, magnesium chloride, and magnesium acetate.

In a preferred embodiment, the dry powder compositions will comprise about 5% to about 50% mIL-4, with about 10% to about 30% more preferred. The stabilizer is present in this embodiment at a range of from about 10% to about 85%, preferably about 15% to about 80%. In some embodiments the stabilizer is present in the range of about 15% to about 50%. A bulking agent, preferably in the form of an amino acid, is optional in this embodiment. If incorporated, it is included in the range of about 10% to about 25%. Buffers are included in a range of about 10% to about 85%, with about 20% to about 50% preferred. The ratio of buffer to stabilizer is from about 1:8.5 to about 8.5:1, more preferred is about 1:8.5 to about 4:1. Embodiments include buffer to stabilizer ratios of about 1:4 to about 4:1, about 1:4 to about 2:1, about 1:2 to about 2:1, and about 2:3 to about 1:1. In this embodiment the preferred fill range into a capsule or blister is from about 5 mg to about 25 mg dry powder composition, more preferable from about 6 mg to about 15 mg. In this embodiment, an optional amount of a magnesium ion may be added as a stabilizer agent. The ion may be present in an about of from about 1 mM to about 220 mM, more preferably from about 10 mM to about 200 mM. The stoichiometry of magnesium ion to buffer may be 1, 2, 4, 6, 8, or in this embodiment. A preferred mIL-4 molecule in this embodiment is that shown in FIG. 1B. Dosing of an effective amount of the composition of this embodiment may be once, twice, or 3 times a day.

The dry powder compositions of the present invention may be characterized by certain properties, including a glass transition temperature of the composition, temperature at which the compositions can be stored, duration of storage, and their capacity for retaining the original specific activity and protein integrity after the composition is stored.

In some embodiments, the dry powder compositions of the present invention have a glass transition temperature which is, at any storage temperature described below, at least 50° C. higher than a storage temperature at which the composition is stored, preferably at least 75° C. higher than the storage temperature, and even more preferably at least 100° C. higher than the storage temperature.

With respect to the recommended storage temperature, in some embodiments, the storage temperature of the dry powder compositions of the present invention is about room temperature (15-25° C.) or below, such as between about 2° C. and 8° C. In some embodiments, the dry powder compositions of the present invention may be stored between about 2° C. and 8° C. during a first initial portion of the storage period, followed by being stored at room temperature during a second portion of the storage period. Those skilled in the art can determine the duration of the first and second portions of the storage period.

Embodiments of the invention further provide for the total storage period of the dry powder compositions of the present invention of at least two years after the manufacture date of the drug product, during which storage of the composition retains at least 80%, e.g., at least 90%, of the original specific activity after the composition is stored at the storage temperature over a period of three months. In some embodiments, the composition at least 95% of the original specific activity after the expiration of the total storage period, for example, retains at least 98% of the original specific activity.

Some other properties characterizing the dry powder compositions of the present invention include the moisture content, pH values, the degree of aggregation of the protein, and the degree of oxidation of the protein. In some embodiments, the moisture content is between about 1% and about 10%, such as between about 1% and about 5%, for example, between about 1% and about 3%. In an embodiment, the moisture content is less than 1%. The pH value of the dry powder compositions of the invention is generally between about 3 and 6, for example, between about 4 and 5. In alternate embodiments the pH range may be between about 3 and 7 with, for example a pH range of between about 6 and 7. With respect to the degree of aggregation of the protein, the compositions typically show aggregates of about 3% or less after the expiration of the total storage period, such as about 1% or less, for example, about less than 1%.

In some embodiments, the degree of oxidation of the therapeutically active material in the dry powder compositions of the present invention is, relative to the drug substance, about 10% or less, after the expiration of the total storage period of the composition, such as about 5% or less, for example, about 3%.

The dry powder compositions of the present invention can be prepared using any suitable method. Preferred methods include, for example, an aqueous solution of the therapeutically active material containing mIL-4 subjected to a process such as freeze drying, spray drying, or freeze spray drying, and can further optionally include milling or lyophilization with milling. In one preferred procedure, a solution is prepared having a mass concentration of solids between about 1 and 5% solids, of which the fraction of the active component (i.e., mIL-4) is as described above. The remaining fraction of solids comprises other components, such as a buffer, a stabilizing agent, and/or bulking excipients, as the case may be.

The solution is then directed through a nozzle that is set at a specific pressure and temperature to create droplets. The droplets enter a chamber established at a specified temperature to dry. The dry particles of a specific size range are collected in a cyclone. This powder is then filled into a primary container at a specified fill weight. The primary container can be any suitable container that provides for storage at a specified fill weight and provides for release of the material contained therein into an inhaler. A particularly preferred embodiment of the primary storage container is a capsule which can be punctured or broken after it has been inserted into an inhaler. Those having ordinary skill in the art can determine the pressure and temperature to be used to form the droplets as well as the drying temperature.

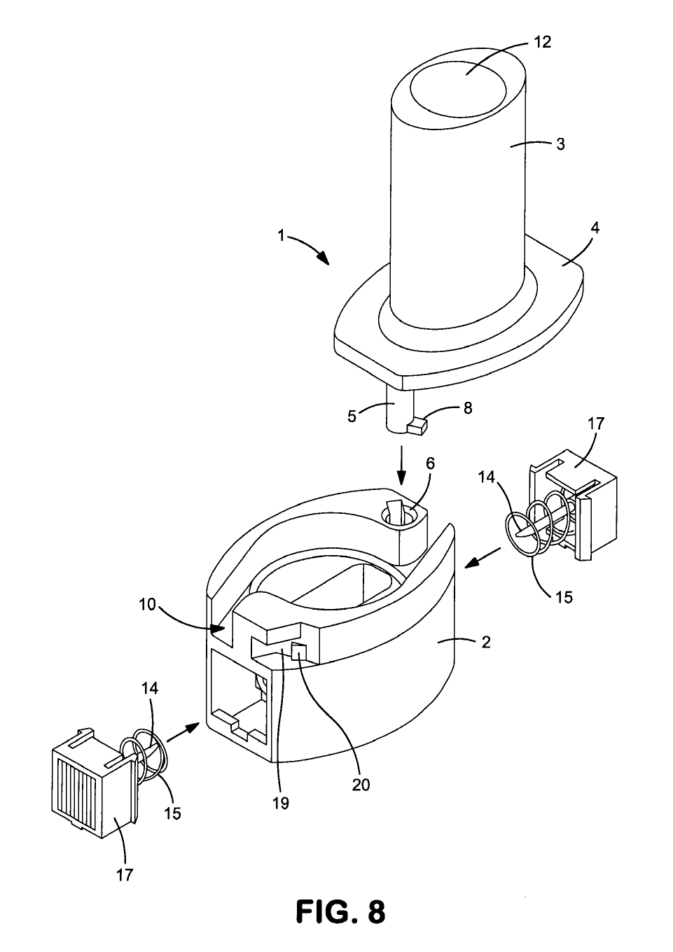

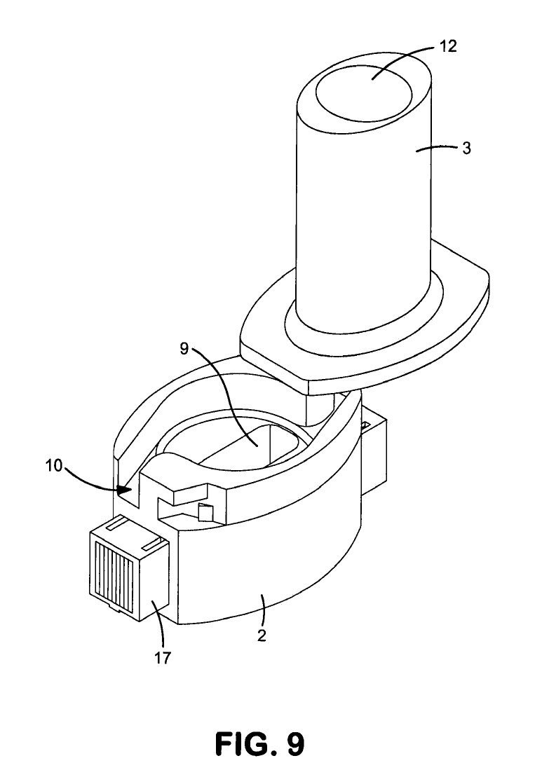

The powder may be inhaled using a suitable inhaler device, e.g., from RS01 Model 7 Inhaler, as described in U.S. Patent application No. 2003-0000523 and in corresponding European patent application No. EP 1270034. The inhaler is designed to ensure that when the powder according to any embodiment of the present invention is inhaled by the patient, the emitted dose of the composition is about 70 mass % or higher. The inhaler may be described in more detail with the reference to FIGS. 8-13 as follows.

As shown by FIG. 8, the inhaler device 1 comprises an inhaler nosepiece 3, including a flange 4, having a peg 5 which can be engaged in a corresponding hole 6 formed in an inhaler body 2. As shown by FIG. 9, the hole 6 is provided with a longitudinal slot, in which can engage a cross tooth 8 of the peg 5, and a bottom ring-like recess (not shown), in which the tooth 8 can slide. Thus, it is possible to engage the peg 5 in the hole 6, by causing the tooth 8 to pass through the longitudinal slot and, upon achieving the bottom, it is possible to fully rotate the peg 5 in its hole 6, thereby also rotating the inhaler nosepiece 3 with respect to the inhaler body 2.

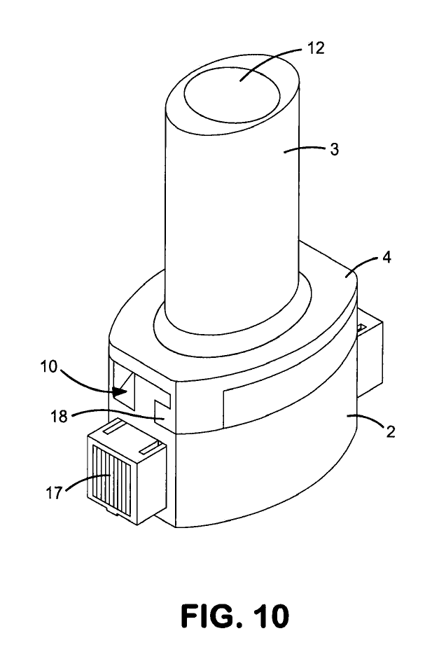

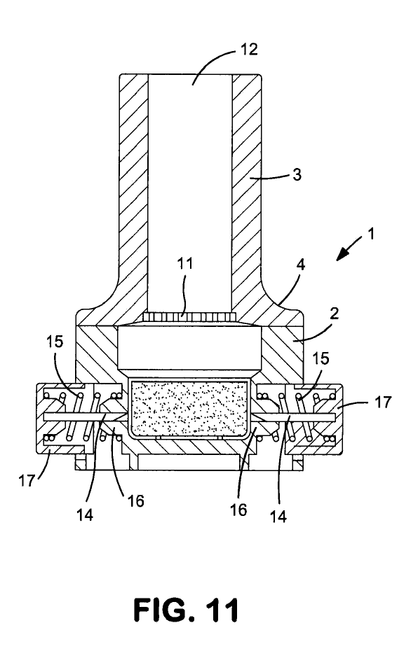

The inhaler nosepiece 3 can be locked in its closed condition, as further shown on FIGS. 8-13, by a snap type of locking means, including a hook portion 18 (FIG. 10) of the flange 4 having a small ridge (not shown), for engaging a corresponding ridge 20 (FIG. 8) formed inside a latching recess 19 (FIG. 8), defined in the inhaler body 2. The inhaler body 2 is further provided with a recess for the capsule, the recess being upward opened and communicating with the outside through a perforated plate or grid 11 (FIGS. 11-13), included in the inhaler nosepiece 3 at the flange 4 and designed for separating the capsule recess 9 (FIG. 9) from the duct 12 (FIG. 9) of the nosepiece 3.

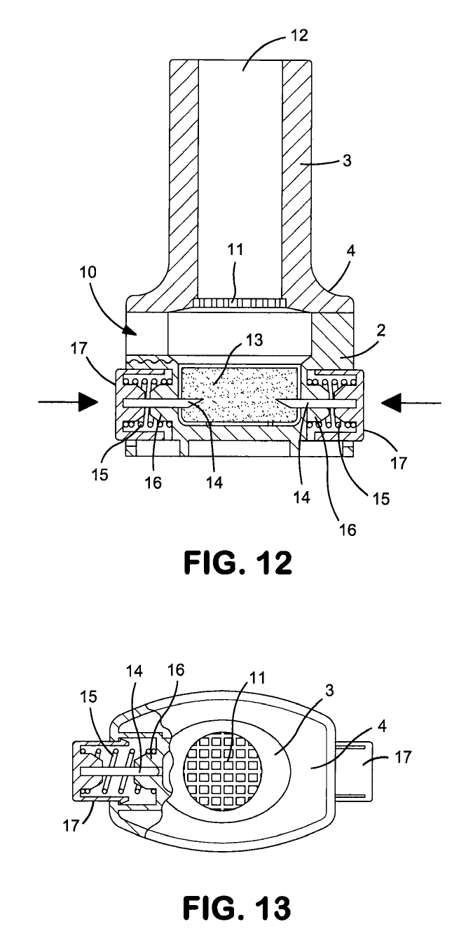

The capsule 13 (FIG. 12) can be engaged in the recess 9, the capsule being of a known type and adapted to be perforated to allow the drug contents held therein to be easily accessed, wherein the perforating operation being performed by any suitable perforating means. In the shown inhaler 1, the perforating means comprise a pair of perforating needles 14 (FIGS. 8 and 11-13) which can transversely slide as counter-urged by resilient elements comprising, in this embodiment, coil springs 15 (FIGS. 8 and 11-13), coaxial with the perforating needles 14 and operating between respective abutment element 16 (FIGS. 11-13), rigid with the inhaler body 2, and a hollow push-button element 17 (FIGS. 8-13). The perforating needles 14 are similar to hypodermic needles and have a beveled tip, for facilitating said perforating needles 14 in perforating the coating of the capsule 13.

The operation of the inhaler device 1 may be further described as follows. In the open condition, as shown on FIG. 9, a capsule is engaged in the capsule recess 9 and the nosepiece 3 is snap closed on the inhaler body 2. By pressing the push-button elements 17, the perforating needles 14 will be forced to perforate the capsule 13, thereby its contents, such as a dry powder of the present invention, will be communicated with the capsule recess. By applying suction on the nosepiece 3, an air flow will be generated which, coming from the outside through the holes 10, will enter the capsule recess, thereby mixing with the capsule contents and, passing through the grid 11 and duct 12, will allow the products to be inhaled.

If desired, the inhaler device 1 may employ hypodermic needles as the perforating needles 14. Since this type of needle presents a very small resistance against perforation and a very accurate operation, it is possible to use needles having a comparatively large diameter, without damaging the capsule, thereby providing a very simple perforating operation. The use of small number of perforating needles, such as only two, allows to reduce, the perforated cross section being the same, the contact surface between the needle and capsule, with a consequent reduction of the friction and of the problems affecting the prior inhalers.

The dry powder compositions of the present invention are administered, via inhalation administration, to a patient in need thereof, such as a patient suffering from asthma, or other obstructive lung diseases such as bronchitis, emphysema, bronchiectasis and cystic fibrosis; fibrotic lung diseases such as interstitial pulmonary fibrosis and other interstitial lung diseases of unknown origin; sarcoidosis and miscellaneous respiratory tract conditions such as nasal polyposis and pulmonary eosinophilia and eosinophilic granuloma. The efficiency of administration can be characterized by the emitted dose of the composition, when inhaled by the patient, and by the deposited fraction of the particles having a particular size. According to embodiments of the present invention, when dry powder compositions of the invention are so inhaled, the emitted dose of the composition is about 70 mass % or higher, such as about 80 mass % or higher, for example, about 90 mass % or higher. Due to practical limitations, the emitted dose typically does not exceed 90 mass % or 95 mass %. According to embodiments of the present invention, when dry powder compositions of the invention are so inhaled, the deposited fraction of the particles entering the lung and having the value of diameter not exceeding about 5 μm is between about 25 and about 60 mass %, such as between about 40 and about 60 mass %, for example, between about 50 and about 60 mass %. In one embodiment, the minimum deposited fraction of the particles having the value of diameter not exceeding about 5 μm can be about 25 mass %; in other embodiments such minimum deposited fraction can be about 40 mass %, or about 50 mass %. In one embodiment, the maximum deposited fraction of the particles having the value of diameter not exceeding about 5 μm can be about 60 mass %; in other embodiments such maximum deposited fraction can be about 70 mass %, or about 80 mass %. To provide further guidance, some methods of fabricating the dry powder compositions of the present invention are described below in the “Examples” portion of the application.

The dry powder compositions of the present invention are administered to a patient in need thereof for treating and/or preventing various disorders (including allergic disorders), diseases, and pathologies. A preferred example of such a disorder is asthma. Examples of other disorders include other obstructive lung diseases such as bronchitis, emphysema, bronchiectasis and cystic fibrosis; fibrotic lung diseases such as interstitial pulmonary fibrosis and other interstitial lung diseases of unknown origin; sarcoidosis and miscellaneous respiratory tract conditions such as nasal polyposis and pulmonary eosinophilia and eosinophilic granuloma. The kind of delivery to be used is inhalation.

Although the invention describes various dosages, it will be understood by one skilled in the art that the specific dose level and frequency of dosage for any particular subject in need of treatment may be varied and will depend upon a variety of factors. These factors include the activity of the specific polypeptide or functional fragment thereof, the metabolic stability and length of action of that compound, the age, body weight, general health, sex, diet, mode and time of administration, rate of excretion, drug combination, the severity of the particular condition, and the host undergoing therapy.

Generally, however, a typical dosage of mIL-4 will be about 0.005 to 1 mg/kg. For example, for administration of mIL-4, an approximate nominal dosage by aerosol inhalation would be about 0.3 mg to 60 mg, such as about 0.3 mg to about 0.7 mg, or about 0.6 mg to about 1.1 mg, or about 0.9 mg to about 1.6 mg, or about 1.4 mg to about 1.9 mg, or about 1.8 mg to about 2.3 mg, or about 2.2 mg to about 2.8 mg, or about 2.7 mg to about 3.2 mg, or about 3.1 mg to about 4.2 mg, or about 4.1 mg to about 5.2 mg, or about 5.1 mg to about 7.7 mg, or about 7.4 mg to about 10.2 mg, or about 10.1 mg to about 15.2 mg, or about 15.1 mg to about 20.2 mg, or about 20.1 mg to about 25.2 mg, or about 25.1 mg to about 30.2 mg, or about 30.1 mg to about 35.2 mg, or about 35.1 mg to about 40.2 mg, or about 40.1 mg to about 45.2 mg, or about 45.1 mg to about 50.2 mg, or about 50.1 mg to about 55.2 mg, or about 55.1 mg to about 60 mg.

Approximate dosages include, but are not limited to, about 0.3 mg, about 0.5 mg, about 1.0 mg, about 1.5 mg, about 2 mg, about 2.5 mg, about 3.0 mg, about 4 mg, about 5 mg, about 7.5 mg, about 10 mg, about 15 mg, about 20 mg, about 25 mg, about 30 mg, about 35 mg, about 40 mg, about 45 mg, about 50 mg, about 55 mg or about 60 mg, to a subject. Dosages can be administered three times a day, twice daily, daily, every two days, every three days, twice per week, weekly, or as needed. Treatment by administration of mIL-4 may span days, weeks, years, or continue indefinitely, as symptoms persist.

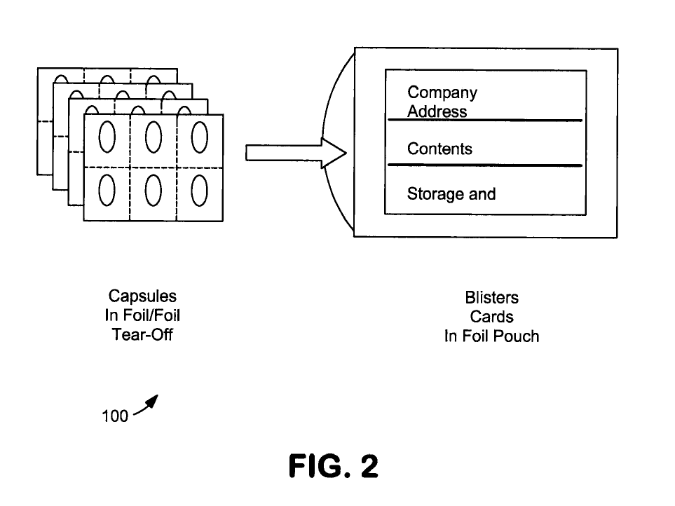

For efficient administration of the compositions of the present invention, a delivery kit schematically shown on FIG. 2 can be used. The kit 100 includes an inhaling element for inhalation by the patient, and a storage container for storing the dry powder composition. The storage container further comprises a primary capsule and a secondary storage container, and further includes an optional label affixed to the storage container to provide the patient with instructions for use. The primary capsule is configured to fit the inhaling element.

In the kit 100 shown by FIG. 2, the dry powder composition of the invention can be in form of capsules not shown, each capsule containing a fill weight nominally between about 5 and 25 mg of the composition, such as between about 5 and about 20 mg, for example, between about 10 and about 20 mg of the composition.

Various sizes and materials can be used as the primary packaging for the drug product. In kit 100 the preferred container is a #3 size capsule. However, other size capsules could be also used, such as size 1, 2 and 4 capsules. Any capsule can be made from various materials, for example, hydroxylpropylmethylcellulose (HPMC) or gelatin. Furthermore, other forms of primary packaging could be used, such as a blister pack. A blister pack arrangement is comprised of a mix of individually openable blisters, of the type in which a base foil formed with blisters is connected to a substantially flat lid foil with the medicant contained within the blister.

Choice of the inhalation device is also critical to the anatomic deposition of the powder, the stability and efficiency of delivery of the dry powder formulation, and for assuring the patient's compliance when using the device. The preferred embodiment is the RS01 Model 7 Inhaler described above. However, various other inhalation devices could be used which would produce different characteristics.

Single dose devices using capsules, like the RSO1, might be employed such as the Aventis Eclipse®, Boehringer Ingelheim Aerohaler® or HandiHaler®. Furthermore, blister pack devices such as the Vectura Aspirair®, or GSK DiskHaler® or Diskus could be substituted. Additionally reservoir devices such as the Orion Pharma Easyhaler®, Inovata Biomed Clickhaler® or Sofotec Novalizer® could accommodate multiple doses of mIL-4. For all inhalation devices, about a 30-day supply will be dispensed to minimize patient misuse, reduce cost to the patient, maintain stability and the safe use of the medicant.

The following examples are intended to further illustrate but not limit the scope of the invention.

### Example 1

### Preparation of Formulations

Four formulations, each comprising the active ingredient mIL-4, were prepared by combining the components into a solution, as shown in Table 1. Subsequent tests and evaluations discussed in the examples that follow refer to them as Formulations 1-4, as shown in Table 1.

TABLE 1
Summary of mIL-4 Containing Formulations

| | | | Sodium | | Sodium |
|---|---|---|---|---|---|
| Formulation | | mIL-4 | Citrate | Sucrose | Lactate |
| # | Formulation | (% w/w) | (% w/w) | (% w/w) | (% w/w) |
| 1 | Citrate pH 5.0 | 88 | 12 | | |
| 2 | Citrate/Sucrose | 54 | 12 | 34 | |
| | pH 5.0 | | | | |
| 3 | Lactate pH 4.0 | 98 | | | 2 |
| 4 | Lactate/Sucrose | 54 | | 44 | 2 |
| | pH 4.0 | | | | |

### Example 2

### Method of Making Dry Powder

The spray drying parameters used to manufacture formulations 1-4 are summarized in Table 2.

TABLE 2
Parameters Used for Spray Drying mIL-4 Formulations

| Parameter | Setting |
|---|---|
| Equipment | Buchi B-191 with Std Cyclone |
| Nozzle Size | 0.7 mm |
| Nozzle Cooling | 5° C. |
| Feedstock Cooling | Ice bath |
| Spray rate (g/min) | Approximately 4.7 |
| Inlet Temperature (° C.) | 100-115 (steady state 108) |
| Outlet temperature (° C.) | 60 |
| Air flow setting | 85% aspiration |
| Nozzle Gas Flow | Inlet Regulator setting of |
| | 95 psi and flow control |
| | reading 750 mm |
| Secondary drying shelf temperature (° C.) | 20 |
| Secondary drying vacuum pressure (mtorr) | <500 |
| Secondary drying duration (hours) | 2 |

### Example 3

### Studies of Mass of Active Component in Composition and Aggregation of the Protein

The content and purity of each formulation was determined by analyzing approximately 20 mg of powder by a RP-HPLC assay method. Three determinations were performed for each powder and the percent by weight was calculated. Table 3 shows the assay results for each formulation. The results are consistent with the theoretical values.

TABLE 3
Summary of Assay Data Obtained on mIL-4 Formulations

| | | Theoretical | | |
|---|---|---|---|---|
| Formu- | | mIL-4 | Mean mIL 4 | Mean mIL-4 |
| lation | | Concentration | Concentration | Concentration |
| No. | Composition | (% w/w)¹ | (% w/w)² | (% w/w)³ |
| 1 | mIL-4/Citrate | 88 | 86 | 79.8 |
| 2 | mIL-4/Citrate/ | 54 | 54 | 51.0 |
| | Sucrose | | | |
| 3 | mIL-4/Lactate | 98 | 94 | 90.6 |
| 4 | mIL-4/Lactate/ | 54 | 46 | 39.9 |
| | Sucrose | | | |

¹Based on ~2% mIL-4 solids content prepared aqueous solutions.
²By HPLC analysis at site 1 (n = 3 determinations).
³By HPLC analysis at site 2 (n = 3 determinations).

The greatest deviation was observed in Formulation 4 where the measured concentration was approximately 8% lower than the expected concentration. The degradation profile was characterized using a reverse phase HPLC degradation method. No noticeable signs of degradation were observed for the spray dried powders other than for Formulation 4 which showed an additional peak indicating aggregation at about 7.6 minutes. The chromatogram obtained is presented on FIG. 1.

### SDS PAGE (Aggregation of the Protein)

Samples were incubated with the anionic detergent sodium dodecyl sulfate. The proteins were separated under non-reducing conditions in a polyacrylamide gel with defined pore size (e.g. ExcelGel SDS, 15% Polyacrylamid, Pharmacia). The separation is proportional to the molecular weight of the proteins. After staining with Coomassie Blue, the gels were scanned (e.g. Scanner JX-330, Sharp) and the number of the individual bands determined. The SDS PAGE evaluation of the protein was conducted for all 4 formulations. The results obtained are presented in FIG. 2. A faint band was observed in spray dried Formulation 4 suggesting some minimal protein decomposition.

### Moisture Content

The moisture content of each formulation was determined using the Karl Fisher titration method. Approximately 10 mg of each formulation was dissolved in 10 mL of Karl Fisher reagent. Samples were analyzed by injecting 1 mL of sample solution into the coulometric cell. Typically three samples were analyzed for each formulation and sample solutions were injected in triplicate. Blanks consisting of the Karl Fisher reagent were used.

Table 4 provides the data regarding the moisture content of each formulation.

TABLE 4
Moisture Content of mIL-4 Formulations

| | Mean Percent Moisture | | | |
|---|---|---|---|---|
| | (mass %) | | | |
| | Formulation | Formulation | Formulation | Formulation |
| | 1 | 2 | 3 | 4 |
| Sample | (mIL-4/ | (mIL-4/Citrate/ | (mIL-4/ | (mIL-4/Lactate/ |
| No. | Citrate)¹ | Sucrose) | Lactate) | Sucrose) |
| Overall | 1.53 | 1.70 | 0.58 | 1.10 |
| Mean | | | | |

### Example 4

### Evaluation of Biological Activity TF1 Functionality Bioassay

The proliferative response of TF-1 cells to IL-4 or IL-13 was used to assess the functional antagonist activity of mIL-4. The TF-1 line was derived from a non-adherent erythroleukemia and is used extensively as a model system because the cells proliferate in response to a number of inflammatory cytokines including IL-4 and IL-13. TF-1 cells were cultured with and without IL-4, IL-13 and mIL-4. The concentration of mIL-4(log nM) versus the mean relative fluorescence units is plotted and data extracted to determine 50% antagonist effect. The EC50 of mIL-4(50% inhibitory effect) for IL-4 and IL13 is reported as a mean and the 95% confidence interval is reported.

The results of the TF1 functionality bio-assay are presented in Table 5. The results indicate there was no loss of activity in the spray dry powder samples due to spray drying.

TABLE 5
Summary of TF1 Data Spray Dried Powders

| Formulations | Mean EC50, nM | 95% Confidence Intervals |
|---|---|---|
| mIL-4 Standard | 0.5237 | 0.3925 to 0.6988 |
| 1-Spray Dry Powder | 0.4957 | 0.3999 to 0.6144 |
| 2-Spray Dry Powder | 0.5177 | 0.4305 to 0.6226 |
| 3-Spray Dry Powder | 0.5768 | 0.4942 to 0.6731 |
| 4-Spray Dry Powder | 0.5552 | 0.4644 to 0.6639 |

### Example 5

### Glass Transition Temperature Measurements

Glass transition temperatures were measured using the method of differential scanning calorimetry (DSC), which was performed using a TA Instruments DSC2920 apparatus using an N₂ flow rate of 10° C. per minute.

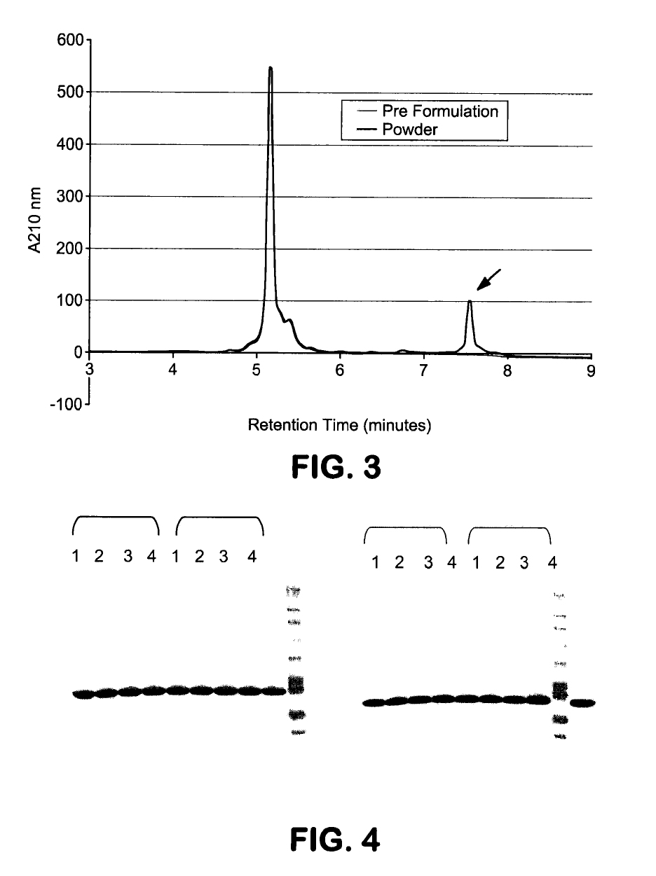

DSC thermal analysis data for the four formulations and the lyophilized starting material are shown by FIGS. 3 and 4. In the range between about 75° C. and 125° C., there are changes in the heat flow indicating glass transition temperatures (50 degrees above the storage temperature).

As can be further observed from FIG. 4, thermograms were obtained from 25° C. to 300° C. The results indicate that the formulations 1 and 2 differ in their thermal behavior because a change in the baseline is observed around 140° C. in the formulations with sucrose (FIG. 3).

### Example 6

### Determining Primary Particle Size Distribution by Laser Diffraction

The geometric particle size distribution of the spray dried formulations was determined using a wet dispersion, laser diffraction method. The Malvern Mastersizer 2000 was used in combination with the Hydro2000S wet cell.

Samples were prepared by weighing approximately 10 to 25 mg of formulation into a 20 mL glass vial. Ten milliliters of dispersant was added to produce a suspension. Samples were sonicated using a probe sonicator at 10% amplitude (40 watts) for 2 minutes to promote particle dispersion. Three samples were prepared for each formulation.

The samples were then analyzed using the Malvern Mastersizer 2000 at the settings shown in Table 6.

TABLE 6
Parameters Used for Determining Geometric Particle Size Distribution

| Instrument Parameter | Setting |
|---|---|
| Analysis Model | General purpose, normal sensitivity, irregular |
| | particle shape, no advanced options |
| Theory | Fraunhofer |
| Sample Measurement Time | 10 seconds |
| Measurement Cycles | 1 |
| Stir Rate | 2,000 rotations per minute |
| Target Obscuration | 10-25% |

The results of the measurements are summarized in Table 7, for Formulations 1 through 4. Only marginal differences in particle size distribution were observed despite the differences in formulation composition. Median diameters ranged from 2.3 to 2.8 μm. Mean D90 values ranged from 4.4 μm to 4.9 μm. Mean SPAN values, a measure of the width of the particle distribution, were less than or equal to 1.4 indicating the particles were relatively monodisperse.

TABLE 7
Mean (% RSD) Malvern Results For Formulations 1-4

| | Formulation Number | | | |
|---|---|---|---|---|
| Parameter | 1 | 2 | 3 | 4 |
| D10 (μm) | 1.5 (0.2%) | 1.2 (0.9%) | 1.4 (0.3%) | 1.3 (2.2%) |
| D50 (μm) | 2.8 (0.2%) | 2.3 (0.5%) | 2.6 (0.3%) | 2.6 (3.1%) |
| D90 (μm) | 4.9 (0.2%) | 4.4 (0.4%) | 4.5 (0.3%) | 4.7 (3.9%) |
| SPAN | 1.2 (0.1%) | 1.4 (0.6%) | 1.2 (0.0%) | 1.3 (1.9%) |

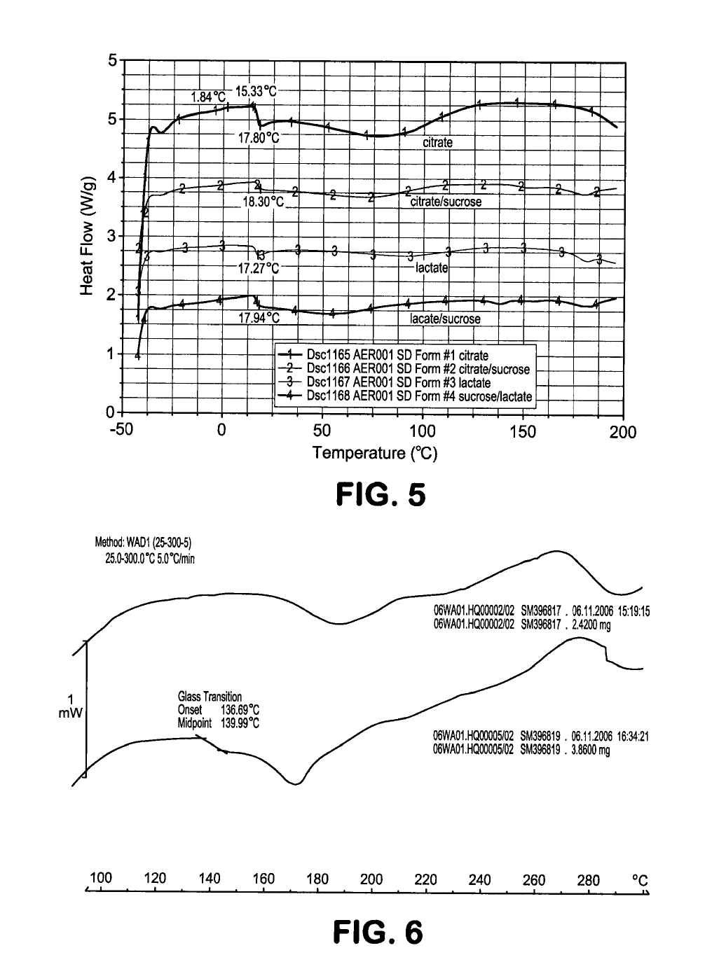

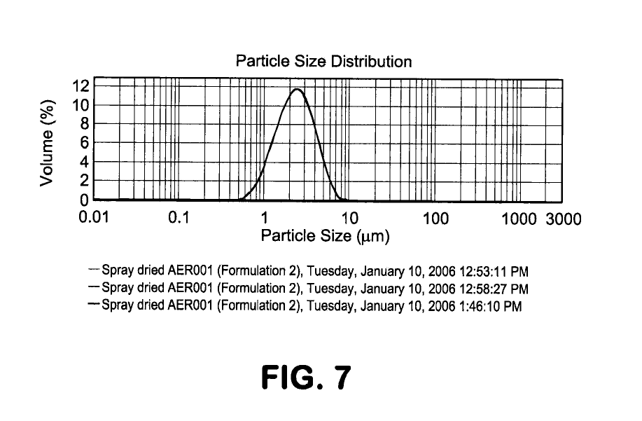

The results for Formulation 2 (mIL-4/Citrate/Sucrose) prepared with 0.1% v/v lecithin in n-octane as the dispersant) are shown by FIG. 5, where the determination was made by laser diffraction immediately after sample delivery to the wet cell. Overlay of 3 determinations is shown by FIG. 5.

### Example 7

### Determining Aerodynamic Particle Size Distribution by Next Generation Impactor

The aerodynamic performance of each formulation was determined using the Next Generation Impactor (NGI). For aerosolization of the formulation, the Plastiape RS01 Model 7, a low resistance capsule device was used. Formulations were filled into size 3 hydroxypropylmethylcellulose (HPMC) capsules from Shinogi.

Nominally, about 5 mg of formulation was weighed into each capsule. Capsule filling and weighing was performed in a conditioned glove box (less than 5% relative humidity, at a temperature of between 15° C. and 25° C.). Capsules were loaded into the device, pierced and sampled into the NGI immediately after preparation.

The flow rate corresponding to a 4 kPa pressure drop was determined using a volumetric flow meter. A modified adapter connecting the inhaler to the USP IP allowed for a direct measurement of the pressure drop. NGIs were setup without a pre-separator. A Copley Critical Flow Controller was setup to draw 4 liters of air through the device during testing. NGI samples were analyzed using the RP HPLC assay method. An air flow rate of approximately 100 L/min was required to produce a 4 kPa pressure drop across the inhaler device. This flow rate was used in all NGI tests.

Table 8 lists the performance parameters calculated for these experiments. The mean fine particle dose (FPD) varied due to the differences in mIL-4 concentration amongst the formulations. When normalized as fine particle fraction (FPF), Formulation 2 had the highest FPF at 96%, followed by Formulations 1 and 3 at 85% and 83%, respectively. Mean MMADs approximated the D50 values determined by Malvern (see Table 7). These results indicate that Formulations 1, 2 and 4, in combination with the inhaler device, are appropriate for inhalation delivery.

TABLE 8
Summary of NGI Results for Formulations 1, 2 and 4

| | Formulation 1 | Formulation 2 | Formulation 4 |
|---|---|---|---|
| | (mIL-4/ | (mIL-4/Citrate/ | (mIL-4/Lactate/ |
| Parameter | Citrate) | Sucrose) | Sucrose) |
| Mean Capsule Fill | 5.0 | 5.2 | 5.5 |
| Weight (mg) | | | |
| Mean mIL-4 Mass | 4.1 | 2.8 | 2.6 |
| Per Capsule (mg)^{a} | | | |
| Mean FPF < 5 μm | 85 | 96 | 83 |
| (%)^{c} | | | |
| Mean MMAD | 2.2 | 2.0 | 2.8 |
| (μm) | | | |
| Mean GSD | 2.4 | 1.6 | 1.5 |

^{a}theoretical mass determined as cap fill wt × mIL-4 concentration per formulation.

### Example 8

### Determining Emitted and Fine Particle Dose

The data for the emitted mass and fine particle dose (deposition) results are summarized in Table 9 using different fill weights for Formulation 1 manufactured at two different primary particle sizes (Batch 1 with a mean particle size of 4.8 μm and Batch 2 with a mean particle size of 3.3 μm Data for the emitted mass and fine particle dose (deposition) results are summarized in Table 10 using different fill weights for Formulation 2 manufactured at two different primary particle sizes (Batch 1 with a mean particle size of 2.9 and Batch 2 with a mean particle size of 4.2 μm)). As can be seen in the results, the fill weight impacts both the emitted mass and the fine particle fraction.

TABLE 9
Deposition Results for Different Fill
Weights (Formulation 1 Batches)

| | | | Formulation 1 | | | Formulation 1 | | |
|---|---|---|---|---|---|---|---|---|
| Test | | Units | Batch 1 | | | Batch 2 | | |
| Primary | | μm | D50 = 4.8 μm | | | D50 = 3.3 μm | | |
| Particle Size | | | | | | | | |
| of Bulk Powder | | | | | | | | |
| Assay of | | % | 83.2 | 83.2 | 88.1 | 81.0 | 81.0 | 88.4 |
| Protein | | Amount | 4.2 | 8.3 | 17.6 | 4.1 | 8.1 | 17.7 |
| | | mIL-4/ | | | | | | |
| | | capsule | | | | | | |
| | | (mg) | | | | | | |
| | | RSD % | 1.4 | 1.4 | 0.7 | 1.7 | 1.7 | 0.5 |
| Capsule Fill | | mg | 5 | 10 | 20 | 5 | 10 | 20 |
| Weight | | powder | | | | | | |
| Gravimetric | | Max % | — | 103 | 101 | — | 103 | 101 |
| Emitted | | Min % | — | 93 | 100 | — | 98 | 100 |
| Mass (%) | | RSD % | — | 3.1 | 0.7 | — | 2.2 | 0.7 |
| | | | | (n = | (n = | | (n = | (n = |
| | | | | 10) | 2) | | 10) | 2) |
| NGI | Emitted | Avg mg | 3.5 | 7.5 | 15.3 | 3.3 | 7.0 | 14.6 |
| Re- | dose | mIL-4 | | | | | | |
| sults | (sum of | FPD/ED | 84 | 90 | 87 | 82 | 86 | 83 |
| | stages) | % | | | | | | |
| | | RSD % | 2 | 5 | 9 | 1 | 3 | 4 |
| | FPD | Avg mg | 1.1 | 2.5 | 5.1 | 2.3 | 4.1 | 7.4 |
| | less | mIL-4 | | | | | | |
| | than | FPD/ED | 31 | 34 | 33 | 69 | 59 | 51 |
| | 5 um | % | | | | | | |
| | size | RSD % | 9 | 6 | 10 | 5 | 6 | 8 |
| | Mass | % | 89 | 94 | 93 | 90 | 92 | 89 |
| | Balance | RSD % | 3 | 7 | 11 | 1 | 3 | 1 |

TABLE 10
Deposition Results for Different Fill
Weights (Formulation 2 Batches)

| | | | Formulation 2 | | | Formulation 2 | | |
|---|---|---|---|---|---|---|---|---|
| Test | | Units | Batch 1 | | | Batch 2 | | |
| Primary | | μm | D50 = 2.9 μm | | | D50 = 4.2 μm | | |
| Particle Size | | | | | | | | |
| of Bulk Powder | | | | | | | | |
| Assay of | | % | 46.6 | 46.6 | 56.5 | 46.1 | 46.1 | 55.8 |
| Protein | | Amount | 2.3 | 4.66 | 11.3 | 2.3 | 4.6 | 11.2 |
| | | mIL-4/ | | | | | | |
| | | capsule | | | | | | |
| | | (mg) | | | | | | |
| | | RSD % | 2 | 2 | <1 | 6 | 6 | 1 |
| Capsule Fill | | mg | 5 | 10 | 20 | 5 | 10 | 20 |
| Weight | | powder | | | | | | |
| Gravimetric | | Max % | — | 102 | 100 | — | 107 | 100 |
| Emitted | | Min % | — | 89 | 99 | — | 97 | 99 |
| Mass (%) | | RSD % | — | 4.6 | 0.7 | — | 2.9 | 0.7 |
| | | | | (n = | (n = | | (n = | (n = |
| | | | | 10) | 2) | | 10) | 2) |
| NGI | Emitted | Avg mg | 2.0 | 3.7 | 9.2 | 2.0 | 4.4 | 9.8 |
| Re- | dose | mIL-4 | | | | | | |
| sults | (sum of | FPD/ED | 85 | 80 | 82 | 88 | 96 | 88 |
| | stages) | % | | | | | | |
| | | RSD % | 2 | 4 | 3 | 4 | 4 | 3 |
| | FPD | Avg mg | 1.3 | 2.6 | 4.8 | 0.7 | 1.6 | 4.0 |
| | less | mIL-4 | | | | | | |
| | than | FPD/ED | 66 | 71 | 52 | 35 | 36 | 41 |
| | 5 um | % | | | | | | |
| | size | RSD % | 7 | 12 | 6 | 26 | 11 | 4 |
| | Mass | % | 94 | 85 | 88 | 93 | 100 | 90 |
| | Balance | RSD % | 2 | 1 | 3 | 2 | 3 | <1 |

### Example 9

### Studies of Storage Conditions and Stability

Four formulations were evaluated for their physical and chemical properties after being spray dried under similar conditions. All formulations had geometric particle size distributions, as determined by laser diffraction, satisfactory for inhalation delivery. Mean particle size diameter (D50) values ranged from 2.3 to 2.8 μm and mean 90^{th} percentile diameter (D90) values ranged from 4.4 to 4.9 μm. The moisture content of all formulations was less than 2% by mass. However, the moisture absorption profile for formulation 3 showed a distinct weight loss at 30-40% RH indicating crystallization. The formulation was eliminated from further testing.

The aerodynamic performance of formulations 1, 2 and 4 appeared suitable for inhalation delivery. However, chemical analysis of formulation 4 indicated the presence of an unknown peak by RP-HPLC. Thus, formulations 1 and 2 were considered for 3 month stability testing at 5° C. and 30° C./65% RH.

The stability data results in Table 11 pertain to a new batch of dry powder similar to Formulation 2, i.e., 75% mIL-4, 15% sucrose and 10% citrate, pH 5.0.

TABLE 11
mIL-4 Bulk Inhalation Dry powder - Stability results

| | | | | 1 month | | 1 month | | 3 month | | 3 month | |
|---|---|---|---|---|---|---|---|---|---|---|---|
| Test name | Specification | T0 | | (5° C.) | | (30° C./65%) | | (5° C.) | | (30° C./65%) | |
| Description | White powder, | White powder, | | White powder, | | White powder, | | White powder, | | White powder, | |
| | without | without | | without | | without | | without | | without | |
| | aggregates and no | aggregates and no | | aggregates and no | | aggregates and no | | aggregates and no | | aggregates and no | |
| | foreign particles | foreign particles | | foreign particles | | foreign particles | | foreign particles | | foreign particles | |
| Identification (by | The main peak RT | The main peak RT | | The main peak RT | | The main peak RT | | The main peak RT | | The main peak RT | |
| HPLC) | of sample is | of sample Should be | | of sample is | | of sample is | | of sample is | | of sample is | |
| | within ±1 min of | within ±1 min of | | within ±1 min of | | within ±1 min of | | within ±1 min of | | within ±1 min of | |
| | the main peak RT | the main peak RT | | the main peak RT | | the main peak RT | | the main peak RT | | the main peak RT | |
| | of reference | of reference | | of reference | | of reference | | of reference | | of reference | |
| | standard | standard | | standard | | standard | | standard | | standard | |
| Water | Not more than | 3.0% | w/w | 2.8% | w/w | 3.0% | w/w | 3.0% | w/w | 2.8% | w/w |
| | 3.0% w/w | | | | | | | | | | |
| Content of (by | Not less than 0.68 | 0.74 | mg/mg | 0.71 | mg/mg | 0.71 | mg/mg | 0.70 | mg/mg | 0.70 | mg/mg |
| UV) Protein | mg/mg and not more | | | | | | | | | | |
| | than 0.83 mg/mg of | | | | | | | | | | |
| | WA01/powder, | | | | | | | | | | |
| | calculated with | | | | | | | | | | |
| | reference to the | | | | | | | | | | |
| | anhydrous basis | | | | | | | | | | |
| Microbial limit | Not more than 100 | Less that 10 | | NT | | NT | | <10 | cfu/g | <10 | cfu/g |
| test Total aerobic | cfu/g | cfu/g | | | | | | | | | |
| count | | | | | | | | | | | |
| Microbial limit | Not more than 10 | 0 | cfu/g | NT | | NT | | <10 | cfu/g | <10 | cfu/g |
| test Total yeast | cfu/g | | | | | | | | | | |
| and molds count | | | | | | | | | | | |
| Staphylococcus | Absence | Absent | | NT | | NT | | Absent | | Absent | |
| aureus | | | | | | | | | | | |
| Pseudomonas | Absence | Absent | | NT | | NT | | Absent | | Absent | |
| aeruginosa | | | | | | | | | | | |
| Enterobacteria | Absence | Absent | | NT | | NT | | Absent | | Absent | |
| and other gram- | | | | | | | | | | | |
| negative micro- | | | | | | | | | | | |
| organisms | | | | | | | | | | | |
| Bacterial | Not more than 10 | Less than 10 | | NT | | NT | | <10 | EU/mg | <10 | EU/mg |
| Endotoxins | EU/mg | EU/mg | | | | | | | | | |
| Particle size d | Not less than 2.5 μm | 2.8 | μm | NT | | NT | | 2.6 | μm | 2.6 | μm |
| (0.5) | and not more than 3.4 | | | | | | | | | | |
| | μm | | | | | | | | | | |
| Completeness of | Upon reconstitution | Clear and | | NT | | NT | | Clear and | | Clear and | |
| solution | (25 mg/ml in water): | transparent solution | | | | | | transparent solution | | transparent solution | |
| | Report result | without suspended | | | | | | without suspended | | without suspended | |
| | | particles | | | | | | particles | | particles | |
| pH | Upon reconstitution | 5.2 | | NT | | NT | | 5.0 | | 5.1 | |
| | (25 mg/ml in water): | | | | | | | | | | |
| | Report result | | | | | | | | | | |
| % WA01 Main | Report result | 76.0% | (area) | 75.2% | (area) | 76.7% | (area) | 75.7% | (area) | 75.8% | (area) |
| Peak (by HPLC) | | | | | | | | | | | |
| % Pre-Peaks of | Report result | 4.0% | (area) | 3.4% | (area) | 3.9% | (area) | 3.7% | (area) | 3.7% | (area) |
| WA01 (by | | | | | | | | | | | |
| HPLC) | | | | | | | | | | | |
| % Post-Peaks of | Report result | 20.0% | (area) | 21.4% | (area) | 19.3% | (area) | 20.6% | (area) | 20.5% | (area) |
| WA01 (by | | | | | | | | | | | |
| HPLC) | | | | | | | | | | | |

| | | | | | | |
|---|---|---|---|---|---|---|
| Aerosol | Report result | ED/DCU: 84.7% | NT | NT | ED/DCU: 80.8% | ED/DCU: 86.6% |
| Performance (by | | FPF: 78.4% | | | FPF: 75.7% | FPF: 77.4% |
| NGI) | | | | | | |
| SE-HPLC | Conforms if RT of | Conforms | Conforms | Conforms | Conforms | Conforms |
| Identity, | WA01 (AER 001) | | | | | |
| Purity | peak is comparable to | | | | | |
| | RT of the reference | | | | | |
| | standard | | | | | |
| | Report % Main Peak | 100 | 100 | 100 | 100 | 100 |
| | Report % Pre Peak | None Detected | None Detected | None Detected | None Detected | None Detected |
| SDS-PAGE | Migration of the main | Pass | Pass | Pass | Pass | Pass |
| (reducing) | band corresponds to | | | | | |
| | reference standard | | | | | |
| | Report main band | Single Band, | Single Band, | Single Band, | Single Band, | Single Band, |
| | detected and its | 14 KDa | 14 KDa | 14 KDa | 14 KDa | 14 KDa |
| | corresponding | | | | | |
| | molecular weight | | | | | |
| | range (KDa) | | | | | |
| | Report all additional | None Detected | None Detected | None Detected | None Detected | None Detected |
| | bands and their | | | | | |
| | corresponding | | | | | |
| | molecular weight | | | | | |
| | range (KDa) | | | | | |
| SDS-PAGE | Migration of the main | Pass | Pass | Pass | Pass | Pass |
| (non- | band corresponds to | | | | | |
| reducing) | reference standard | | | | | |
| | Report each individual | Single Band, | Single Band, | Single Band, | Single Band, | Single Band, |
| | band detected and its | 14 KDa | 14 KDa | 14 KDa | 14 KDa | 14 KDa |
| | corresponding | | | | | |
| | molecular weight | | | | | |
| | range (KDa) | | | | | |
| | Report all additional | None Detected | None Detected | None Detected | None Detected | None Detected |
| | bands and their | | | | | |
| | corresponding | | | | | |
| | molecular weight | | | | | |
| | range (KDa) | | | | | |
| Functionality | Report EC 50 (nM) | N 1 = 0.2456 | N 1 = 0.1378 | N 1 = 0.1908 | N 1 = 0.3021 | N 1 = 0.3000 |
| by Bioassay | | N 2 = 0.2629 | N 2 = 0.1781 | N 2 = 0.1839 | | |
| (specific | Report 95% | N1 = 0.2059- | N1 = 0.1178- | N1 = 0.1633- | N 1 = 0.2491- | N 1 = 0.2184- |
| activity) | Confidence Interval | 0.2929 | 01611 | 0.2229 | 0.3664 | 0.4122 |
| | EC 50 (nM) | N2 = 0.2131- | N2 = 0.1531- | N2 = 0.1512- | | |
| | | 0.3243 | 0.2032 | 0.2236 | | |

| | | | |
|---|---|---|---|
| Report Standard EC | 0.2644 | 0.1939 | 0.3052 |
| 50 (nM) | 0.2317-0.3018 | 0.1710-0.2200 | 0.2082-0.4474 |
| 95% Confidence | | | |
| Interval (EC 50 nM) | | | |

NT—not tested

### Example 10

### Kit Contents and Packaging

Dynamic Vapor Sorption (DVS) was performed on the spray dried material to evaluate moisture uptake. The samples were analyzed using a SMS DVS 2000 system ramping from 0 to 90% RH with a dM/dT value of 0.001%. Lyophilized mIL-4 was also characterized for reference. The dynamic vapor sorption (DVS) studies of the four spray-dried formulations of IL-4 indicate that the moisture uptake properties of formulation 1, 2 and 4 are consistent to each other and to mIL-4 lyophilized cake. These show an increase in moisture with increasing relative humidity and no other moisture induced events.

Formulation 3 shows a distinct weight loss event at 30-40% RH which can be attributed to a crystallization and potential instability for this formulation. Tables 12 and 13 provide summaries of DVS data.

TABLE 12
Comparison of DVS Properties of mIL-4 Spray-Dried Formulations

| Formu- | Formulation | |
|---|---|---|
| lation # | Components | DVS result |
| 1 | Citrate | Hygroscopic, sorbs 41% moisture by 90% RH, |
| | | retained its opaque solid state during experiment, |
| | | moisture sorption profile consistent with mIL-4 |
| | | lyophilized cake and 2 other spray dried |
| | | formulations |
| 2 | Citrate/ | Hygroscopic, sorbs 56% moisture by 90% RH, |
| | sucrose | formed a transparent solid during experiment, |
| | | moisture sorption profile consistent with mIL-4 |
| | | lyophilized cake and 2 other spray dried |
| | | formulations |
| 3 | Lactate | Hygroscopic, sorbs 8% moisture by 90% RH, |
| | | moisture sorption profile inconsistent with mIL-4 |
| | | lyophilized cake and with 3 other spray dried |
| | | formulations |
| 4 | Lactate/ | Hygroscopic, sorbs 36% moisture by 90% RH, |
| | sucrose | retained its opaque solid state during experiment, |
| | | moisture sorption profile consistent with mIL-4 |
| | | lyophilized cake and 2 other spray dried |
| | | formulations |
| Control | mIL-4 | Hygroscopic, sorbs 41% moisture by 90% RH |
| | lyophilized | formed a hard solid during experiment, |
| | cake, batch | |

TABLE 13
Summary of Hygroscopicity Data

| | Sorption (%) | | | | |
|---|---|---|---|---|---|
| | mIL-4 | | | | Form #4 |
| Target | Lyophilized | Form #1 | Form #2 | Form #3 | (lactate/ |
| RH (%) | Cake | (citrate) | (citrate/sucrose) | (lactate) | sucrose |
| 0.0 | 0.00 | 0.00 | 0.00 | 0.00 | 0.00 |
| 10.0 | 1.82 | 3.65 | 2.52 | 2.07 | 0.19 |
| 20.0 | 2.91 | 4.92 | 3.15 | 2.91 | 0.64 |
| 30.0 | 3.61 | 6.07 | 3.74 | 1.75 | 1.33 |
| 40.0 | 4.51 | 6.39 | 2.96 | −3.11 | 2.47 |
| 50.0 | 6.17 | 8.32 | 2.81 | −9.71 | 4.12 |
| 60.0 | 9.21 | 11.25 | 7.23 | −8.22 | 6.55 |
| 70.0 | 14.37 | 16.04 | 14.26 | −5.76 | 10.59 |
| 80.0 | 23.05 | 23.67 | 26.49 | −1.04 | 18.47 |
| 90.0 | 41.35 | 41.04 | 55.80 | 7.96 | 35.57 |

The moisture uptake of these formulations requires that the packaging configuration used must minimize moisture ingress to the product. Improper packaging and moisture ingress of these dry powders resulted in instability as seen in Tables 14 and 15.

Tables 14 and 15 show stability data for bulk powder and filled capsules from formulations 1 and 2 packaged in foil overwrap and stored at 5° C., 25°/60% RH and 40° C./75% RH for 12 weeks. Also, for each formulation a set of capsule samples were stored in a glass vial but left exposed (no overwrap, no dessicant) to 25° C. and 60% relative humidity for 8 weeks. In both formulations, bulk powder and capsules over the period of 12 weeks stored in foil overwrap showed a substantial increase in moisture demonstrating that the foil overwrap did not protect the drug product from moisture ingress. The data shown for RP-HPLC and SDS PAGE is characteristic of moisture induced degradation. Capsule samples stored unprotected for 8 weeks showed the largest increase in moisture and pronounced degradation. With improved packaging as seen in Table 11, there was no evidence of increasing moisture and no protein degradation under the accelerated storage condition (30° C./65% RH) over the same timeframe.

TABLE 14
Formulation 1: The Effect of Improper Packaging on Protein Integrity

| | | | | | |
|---|---|---|---|---|---|
| | | T = 4 W | T = 12 W | T = 12 W | T = 12 W |
| | | Unprotected | 5° C. Foil | 25° C./60% RH | 40° C./75% RH |
| Parameter | | Capsule | Overwrap and | Foil Overwrap | Foil Overwrap |
| Tested | T = 0 | sample | desiccant | and desiccant | and desiccant |

Formulation 1 Bulk Powder

| | | | | |
|---|---|---|---|---|
| Moisture | 1.8 | 3.8 | 4.9 | 5.6 |
| Content | | | | |
| Purity by | | | | |
| RP-HPLC | | | | |
| % Main Peak | 84.5 | 84.7 | 86.3 | 71.3 |
| % Pre Peak | 3.7 | 3.0 | 2.6 | 2.4 |
| % Post Peak | 11.8 | 13.3 | 11.2 | 26.4 |
| Purity by | Single | Single | First Band, | First Band, |
| SDS-PAGE | Band, | Band, | 14 KDa, | 14 KDa, |
| | 14 KDa | 14 KDa | Second Band, | Second Band, |
| | | | 28 KDa | 28 KDa |
| | | | | Third Band, |
| | | | | >30 KDa |

Formulation 1 Capsule Samples

| | | | | | |
|---|---|---|---|---|---|
| Moisture | 1.2 | 7.2 | 2.5 | 4.0 | 5.0 |
| Content | | | | | |
| Purity by | | | | | |
| RP-HPLC | | | | | |
| % Main Peak | 84.4 | 75.2 | 86.5 | 86.6 | 67.2 |
| % Pre Peak | 3.7 | 2.4 | 2.6 | 2.4 | 3.3 |
| % Post Peak | 11.9 | 22.4 | 10.9 | 11.0 | 29.6 |
| Purity by | Single | First Band, | Single | First Band, | First Band, |
| SDS-PAGE | Band, | 14 KDa, | Band, | 14 KDa, | 14 KDa, |
| | 14 KDa | Second Band, | 14 KDa | Second Band, | Second Band, |
| | | 28 KDa | | 28 KDa | 28 KDa |
| | | Third Band, | | | Third Band, |
| | | >30 KDa | | | >30 KDa |

TABLE 15
Formulation 2: The Effect of Improper Packaging on Protein Integrity

| | | | | | |
|---|---|---|---|---|---|
| | | T = 4 W | T = 12 W | T = 12 W | T = 12 W |
| | | Unprotected | 5° C. Foil | 25° C./60% RH | 40° C./75% RH |
| Parameter | | Capsule | Overwrap and | Foil Overwrap | Foil Overwrap |
| Tested | T = 0 | sample | desiccant | and desiccant | and desiccant |

Formulation 2 Bulk Powder

| | | | | |
|---|---|---|---|---|
| Moisture | 1.4 | 2.6 | 4.0 | 4.3 |
| Content | | | | |
| Purity by | | | | |
| RP-HPLC | | | | |
| % Main Peak | 85.3 | 86.5 | 86.0 | 79.8 |
| % Pre Peak | 3.3 | 2.4 | 2.3 | 2.9 |
| % Post Peak | 11.4 | 11.1 | 11.7 | 17.4 |
| Purity by | Single | Single | First Band, | First Band, |
| SDS-PAGE | Band, | Band, | 14 KDa, | 14 KDa, |
| | 14 KDa | 14 KDa | Second Band, | Second Band, |
| | | | 28 KDa | 28 KDa |
| | | | | Third Band, |
| | | | | >30 KDa |

Formulation 2 Capsule Samples

| | | | | | |
|---|---|---|---|---|---|
| Moisture | 1.9 | 8.4 | 3.8 | 4.6 | ND |
| Content | | | | | |
| Purity by | | | | | |
| RP-HPLC | | | | | |
| % Main Peak | 85.0 | 74.8 | 86.2 | 86.4 | 70.4 |
| % Pre Peak | 3.5 | 3.6 | 2.6 | 2.5 | 2.3 |
| % Post Peak | 11.4 | 21.6 | 11.2 | 11.1 | 27.4 |
| Purity by | Single | First Band, | Single | First Band, | First Band, |
| SDS-PAGE | Band, | 14 KDa, | Band, | 14 KDa, | 14 KDa, |
| | 14 KDa | Second Band, | 14 KDa | Second Band, | Second Band, |
| | | 28 KDa | | 28 KDa | 28 KDa |
| | | | | | Third Band, |
| | | | | | >30 KDa |

### Example 11

### Treatment of Asthma Patients with Dry Powder Formulation

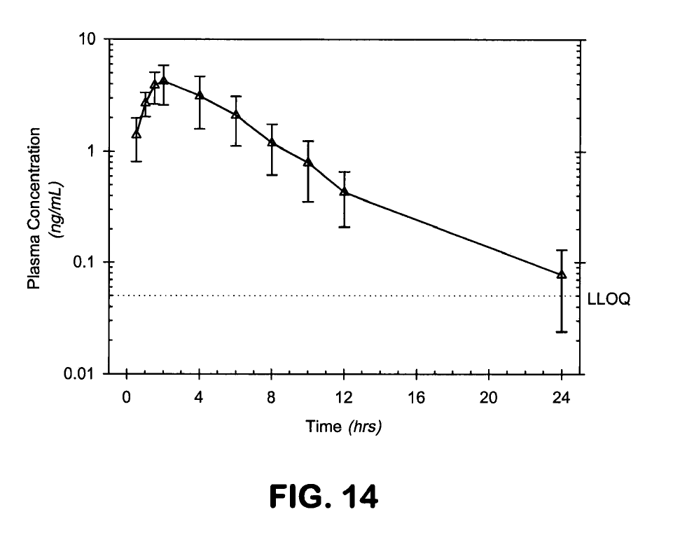

The pharmacokinetics (PK) and local tolerance of inhalation of an IL-4/IL-13 antagonist dry powder composition were determined from a clinical study. Methods: Ten subjects with mild-to-moderate asthma (FM/1% predicted 73-105%, salbutamol reversibility ≧10%) were administered a single dose of 10 mg IL-4 mutein dry powder composition via the inhaler of FIG. 10. The dry powder composition comprised 75% drug load of the mIL-4 in FIG. 1B, 15% sucrose, and 10% citrate (as an approximate ratio of 7:3 sodium citrate to citric acid). Serial measurements of PK and lung function (FEV 1) were collected over a 24-hr period post dosing. Results: Time to peak blood conc. was 2.0 hrs. Half-life in the blood (3-4 hrs) was unaffected by formulation and dose level. Total systemic exposure (AUC0-∞; range 12-39 ng·hr/mL) and between-subject variability (geometric CV on AUC0-∞, 48%) agree with expectations based on in vitro powder and device characterization. Typical changes in FEV1 over 24 hrs were ≦10%, consistent with previous reports of lung function changes following inhalation delivery of drugs (Wilkinson, J. et al. BMJ 1992; 305:931-932). The dry powder composition was well tolerated in asthmatic subjects with no evidence of local irritancy. The mean (±SD) IL-4 mutein plasma concentration versus time profile for the inhaled dry powder composition is shown in FIG. 14.

Although the invention has been described with reference to the above examples, it will be understood that modifications and variations are encompassed within the spirit and scope of the invention. Accordingly, the invention is limited only by the following claims.

What is claimed is:

1. A kit, comprising:
(a) a dispersible powder composition suitable for inhalation by a patient in need thereof, the composition including a therapeutic agent comprising a human interleukin mutein (mhIL-4), a buffer and a stabilizing agent;
(b) an inhaling means for inhalation by the patient, and
(c) a storage means for storing the composition, the storage means comprising a primary container and a secondary storage container,
with the proviso that the primary container is adapted to fit the inhaling means.

2. The kit of claim 1, further comprising patient instructions for use contained in or affixed to the storage means.

3. The kit of claim 1, wherein the primary container is capable of being broken by the inhaling means.

4. The kit of claim 1, wherein the primary container contains between about 5 and 25 mg of the composition.

5. The kit of claim 4, wherein the primary container contains between about 5 and 20 mg of the composition.

6. The kit of claim 5, wherein the primary container contains between about 10 and about 20 mg of the composition.

7. The kit of claim 1, wherein the primary container is a capsule containing between about 5 and about 20 mg of the composition.


SEQUENCE LISTING (4 sequences)

SEQ ID NO: 1 - protein, 129 aa, Homo sapiens [sequence omitted]

SEQ ID NO: 2 - DNA, 387 bp, Homo sapiens
cacaagtgcgatatcaccttacaggagatcatcaaaactttgaacagcctcacagagcagaagactctgtgcaccgagttgaccgtaacagacatctttgctgcctccaagaacacaactgagaaggaaaccttctgcagggctgcgactgtgctccggcagttctacagccaccatgagaaggacactcgctgcctgggtgcgactgcacagcagttccacaggcacaagcagctgatccgattcctgaaacggctcgacaggaacctctggggcctggcgggcttgaattcctgtcctgtgaaggaagccaaccagagtacgttggaaaacttcttggaaaggctaaagacgatcatgagagagaaatattcaaagtgttcgagc

SEQ ID NO: 3 - protein, 130 aa, Artificial sequence
  Synthetic construct
MHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMDEKDSKCSS

SEQ ID NO: 4 - DNA, 396 bp, Artificial sequence
  Synthetic construct
atgcacaaatgcgatatcaccctgcaggaaatcatcaaaaccctgaattctctgaccgaacagaaaaccctgtgcaccgaactgaccgttaccgacatcttcgctgcttcgaaaaacaccaccgaaaaagaaaccttctgccgtgctgctaccgttctgcgtcagttctactctcaccacgaaaaagacacccgttgcctgggtgctaccgctcagcagttccaccgtcacaaacagctgatccgtttcctgaaacgtctggaccgtaacctgtggggtctggctggtctgaacagctgcccggttaaagaagctaaccagtctaccctggaaaacttcctggaacgtctgaaaaccatcatggacgaaaaagactctaaatgctcttcttaataa